(19) United States
(12) Patent Application Publication
(10) Pub. No.: US 2025/0084051 A1
(43) Pub. Date: Mar. 13, 2025

(54) glycerylphosphorylethanolamine (GPE) compound and lipid nanoparticle (LNP)-based drug carrier with same

(71) Applicant: Shandong Academy of Pharmaceutical Sciences, Jinan City (CN)

(72) Inventors: Song XUE, Jinan City (CN); Daizhou ZHANG, Jinan City (CN); Dawei LI, Jinan City (CN); Xuehua YANG, Jinan City (CN); Zhengping LIU, Jinan City (CN); Jie ZHUANG, Jinan City (CN)

(21) Appl. No.: 18/824,028

(22) Filed: Sep. 4, 2024

(30) Foreign Application Priority Data
Sep. 8, 2023 (CN) 202311159656.3

(51) Int. Cl. C07D 339/04; A61K 9/51; C12N 15/88

(52) U.S. Cl. CPC C07D 339/04; A61K 9/5123; A61K 9/5192; C12N 15/88

(57) ABSTRACT

A glycerylphosphorylethanolamine (GPE) skeleton compound and a lipid nanoparticle (LNP)-based drug carrier with the same are provided. The GPE skeleton compound has a structure shown in formula (I):

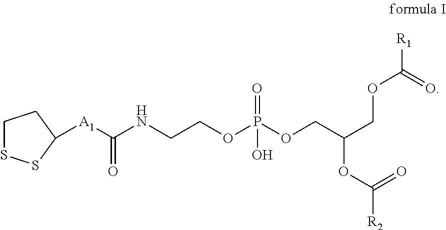

A dithiolane structure is modified on a hydrophilic phosphate terminus of the GPE, such that the dithiolane structure is more fully exposed on a surface of the LNP, and the surface of the LNP can expose more dithiolane, thereby further promoting endocytosis of the LNP by mucosal cells. The GPE skeleton compound has a wide adjustment range in an LNP formulation and can increase a content of the dithiolane in the LNP. A nucleic acid-LNP composition and a pharmaceutical preparation are further provided, and can be used for ocular delivery, pulmonary inhalation delivery, and nasal spray of nucleic acid drugs, providing a new option for extrahepatic delivery of the nucleic acid drugs.

### CROSS REFERENCE TO RELATED APPLICATION

This patent application claims the benefit and priority of Chinese Patent Application No. 202311159656.3, filed with the China National Intellectual Property Administration on Sep. 8, 2023, the disclosure of which is incorporated by reference herein in its entirety as part of the present application.

### TECHNICAL FIELD

A glycerylphosphorylethanolamine (GPE) skeleton compound and a lipid nanoparticle (LNP)-based drug carrier with the same are provided, belonging to the technical field of nucleic acid drug delivery.

### BACKGROUND

Information for disclosing this background section is merely for the purpose of facilitating the understanding of the general background of the present disclosure, and is not necessarily regarded as an acknowledgment or any form of implication that the information constitutes the prior art already known to those of ordinary skill in the art.

Currently, lipid nanoparticles (LNPs) for nucleic acid drug delivery are mainly composed of four components, including cationic/ionizable lipids, neutral auxiliary phospholipids, cholesterol, and PEGylated lipids. These four components can be used to encapsulate nucleic acid drugs through microfluidic devices to obtain the LNPs. At present, three LNP-based nucleic acid drugs are on the market. In 2018, the first LNP-based nucleic acid drug, Patisiran, was successfully launched on the market and became the first siRNA drug for the treatment of hereditary thyroxine-mediated amyloid polyneuropathy. The other two are mRNA vaccines that were conditionally launched on the market during the COVID-19 pandemic. In recent years, billions of doses of LNP-based mRNA drugs have been widely administered and prevented the nCoV-2019 from harming healthy people, proving that LNP technology exhibits desirable safety and excellent immune effects in mRNA drug delivery.

LNP has a high encapsulation rate for nucleic acid drugs, generally above 80%, and can protect nucleic acids from degradation by various enzymes in vivo. However, LNP-based nucleic acid drugs are mostly concentrated in the fields of liver disease and vaccines. Although a large number of nucleic acid drugs are in different stages of clinical trials so far, no nucleic acid drugs targeting other organs have entered clinical research.

Patent CN116212031A disclosed a mucoadhesive lipid material (DSPE-PEG-LA) composed of three parts: lipoic acid, polyethylene glycol, and 1,2-distearoyl-sn-glycero-3-phosphoethanolamine, as well as chemical bonds linking these three parts. This material can be constructed into LNP and further prepared into a nucleic acid drug with mucoadhesive effect. However, the PEG segment has strong flexibility. During the assembly of LNP, the hydrophobic lipoic acid is easily wrapped in an internal hydrophobic core of the LNP, making it difficult for dithiolane to be exposed on the surface of LNP, thereby affecting its function of promoting cell endocytosis. In addition, the preferred dosage of DSPE-PEG-LA in the LNP formulation is generally 1% to 5%, which greatly limits a content range of the dithiolane in LNP.

### SUMMARY

In order to solve the deficiencies of the prior art, an objective of the present disclosure is to provide a glycerylphosphorylethanolamine (GPE) skeleton compound and a lipid nanoparticle (LNP)-based drug carrier with the same. In the present disclosure, a novel compound is designed for the delivery of nucleic acid drugs. The compound has a dithiolane structure, which can react chemically with sulfhydryl groups on the cell surface, thereby promoting the endocytosis of LNP and improving the ability of nucleic acid drugs to enter cells. A dithiolane structure is modified on a hydrophilic phosphate terminus of the GPE liquid. Compared with DSPE-PEG-LA, the GPE lipid with a dithiolane structure can make the dithiolane structure more fully exposed on a surface of the LNP, and the surface of the LNP prepared using the lipid can expose more dithiolane, thereby further promoting endocytosis of the LNP by mucosal cells. The GPE skeleton compound has a wide adjustment range in an LNP formulation and can increase a content of the dithiolane in the LNP. A nucleic acid-LNP composition and a pharmaceutical preparation are further provided, and can be used for ocular delivery, pulmonary inhalation delivery, and nasal spray of nucleic acid drugs, providing a new option for extrahepatic delivery of the nucleic acid drugs.

To achieve the above objective, the present disclosure is achieved by the following technical solutions:

In a first aspect, the present disclosure provides a GPE skeleton compound having a structure shown in formula (I):

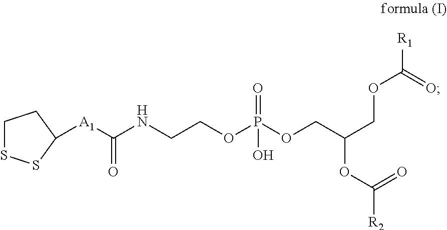

where
R₁ and R₂ are independently selected from the group consisting of C₁₋₂₄ alkyl and C₁₋₂₄ alkenyl, and A₁ is selected from the group consisting of methylene, 1,2-ethanediyl, 1,3-propylenediyl, 1-methyl-1,2-ethanediyl, 1,2-isopropyl, 1-methyl-1,3-propylenediyl, 2-methyl-1,3-propylenediyl, 1,4-butanediyl, 1,5-pentanediyl, 1-methyl-1,4-butanediyl, 2-methyl-1,4-butanediyl, 3-methyl-1,4-butanediyl, 2,2-dimethyl-1,3-butanediyl, 1,6-hexanediyl, 1-methyl-1,5-pentanediyl, 2-methyl-1,5-pentanediyl, 3-methyl-1,5-pentanediyl, 1-ethyl-1,4-butanediyl, and 2-ethyl-1,4-butanediyl.

In a second aspect, the present disclosure provides a preparation method of the GPE skeleton compound in the first aspect, including the following steps:
dissolving a compound shown in formula (II) in chloroform with stirring, adding carbonyl diimidazole, stirring an obtained first mixture in the dark, adding a compound shown in formula (III), subjecting an obtained second mixture to a reaction in the dark, subjecting an obtained product to rotary evaporation to remove the chloroform, and conducting column chromatography to obtain the GPE skeleton compound; where

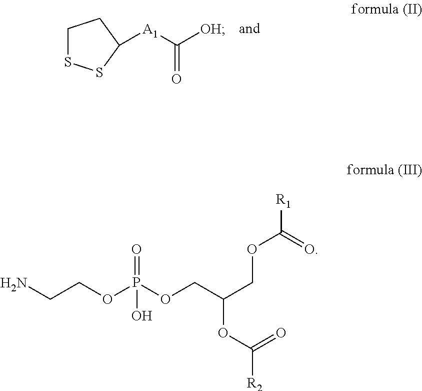

Preferably, the compound shown in formula (II), the carbonyl diimidazole, and the compound shown in formula (III) are at a molar ratio of 1:(0.75-0.85):(1.05-1.15).

Preferably, the reaction is conducted in the dark at 29° C. to 31° C. for 22 h to 26 h.

In a third aspect, the present disclosure provides an LNP-based drug carrier, including a first lipid compound and a second lipid compound, where the first lipid compound is one or more selected from the group consisting of the GPE skeleton compound in the first aspect and a pharmaceutically acceptable salt, a stereoisomer, a tautomer, a solvate, a chelate, and a non-covalent complex thereof, and the second lipid compound is selected from the group consisting of a cationic lipid or an ionizable lipid, a sterol, and an amphiphilic lipid.

Preferably, the cationic lipid or the ionizable lipid is one or more selected from the group consisting of DLinDMA, DODMA, DLin-MC2-MPZ, DLin-KC2-DMA, DOTAP, C12-200, SM-102, and ALC-0315; and
the amphiphilic lipid is one or more selected from the group consisting of PEG-DSPE, PEG-PE, PEG-DMG, PEG-C14, PEG-c-DMA, PEG-modified ceramide, PEG-modified dialkylamine, PEG-modified diacylglycerol, Tween-20, Tween-80, PEG-DPG, PEG-s-DMG, PEG-c-DOMG, and GalNAc-PEG-DSG.

Preferably, the first lipid compound, the cationic lipid or the ionizable lipid, the sterol, and the amphiphilic lipid are at a molar ratio of (2-20):(10-65):(20-55):(0.2-20); and
preferably, the first lipid compound, the cationic lipid or the ionizable lipid, the sterol, and the amphiphilic lipid are at a molar ratio of (5-10):(30-50):(35-55):(0.5-10).

In a fourth aspect, the present disclosure provides a nucleic acid-LNP composition having a particle size of 30 nm to 300 nm and including the LNP-based drug carrier in the third aspect and a nucleic acid drug, where the LNP-based drug carrier and the nucleic acid drug are at a mass ratio of 1:(1-20).

Preferably, the nucleic acid drug is one or more selected from the group consisting of a DNA, an siRNA, an mRNA, a dsRNA, an antisense nucleic acid, a microRNA, an antisense microRNA, antagomir, a microRNA inhibitor, a microRNA activator, and an immunostimulatory nucleic acid.

In a fifth aspect, the present disclosure provides a pharmaceutical preparation, including the nucleic acid-LNP composition in the fourth aspect, a pharmaceutically acceptable buffer salt, an osmotic pressure regulator, and a suspending agent.

According to the above-mentioned technical solutions, the present disclosure has the following advantages:

In the present disclosure, a dithiolane ring in the GPE skeleton compound is located at the hydrophilic phosphate terminus. During the assembly of LNP, the dithiolane ring is exposed to a large extent on the surface of LNP and is more likely to interact with thiol groups on the cell surface.

In the present disclosure, the GPE skeleton compound has a wide adjustment range in the LNP formulation, and can adjust the content of dithiolane in LNP within a large range, thereby promoting the endocytosis of LNP by mucosal cells.

Using eGFP plasmid as a model nucleic acid drug, a nucleic acid-LNP composition based on the GPE skeleton compound has an excellent transfection effect. The pharmaceutical preparation based on the above composition has broad application prospects in pulmonary inhalation administration, ocular administration, and nasal administration of nucleic acids.

In the present disclosure, the GPE skeleton compound has a simple synthesis route, a convenient purification process, and an excellent market prospect.

### BRIEF DESCRIPTION OF THE DRAWINGS

The accompanying drawings which constitute a part of the description of the present disclosure are intended to provide further understanding of the present disclosure. The exemplary examples of the present disclosure and descriptions thereof are intended to explain the present disclosure and do not constitute an inappropriate limitation to the present disclosure.

FIG. 1 shows the particle size distribution of a nucleic acid-LNP composition encapsulating siPLK1 small interfering RNA in Example 14;

FIG. 2 shows the particle size distribution of a nucleic acid-LNP preparation encapsulating siPLK1 small interfering RNA in Example 16;

FIG. 3 shows the distribution of Cy3-labeled LNPs in A549 cells in Example 18, where black is the cell nucleus stained with DAPI, dark gray is the Cy3-labeled LNP, and light gray is the lysosome labeled with Lysotracker green; the left figure shows LNPs containing compound 7, and the right figure shows LNPs without dithiolane structure;

FIG. 4 shows a fluorescence microscopy image of A549 cells transfected with the nucleic acid-LNP composition carrying the cGFP plasmid in Example 19;

FIG. 5 shows the particle size distribution of a nucleic acid-LNP pharmaceutical preparation encapsulating siPLK1 small interfering RNA in Example 20;

FIG. 6 shows a transmission electron microscopy (TEM) image of LNP 1 in Example 21; and

FIG. 7 shows a TEM image of LNP 2 in Example 21.

### DETAILED DESCRIPTION OF THE EMBODIMENTS

Unless otherwise stated, the following terms have the following meanings:

The term “pharmaceutically acceptable salt” refers to a salt of a compound that is substantially non-toxic to humans. Pharmaceutically acceptable salts generally include, but are not limited to, salts formed by reacting the compounds of the present disclosure with pharmaceutically acceptable inorganic/organic acids or inorganic/organic bases. Such salts are also referred to as acid addition salts or base addition salts. Common inorganic acids include (but are not limited to) hydrochloric acid, hydrobromic acid, sulfuric acid, and phosphoric acid. Common organic acids include (but are not limited to) trifluoroacetic acid, citric acid, maleic acid, fumaric acid, succinic acid, tartaric acid, lactic acid, acetic acid, benzoic acid, methanesulfonic acid, benzenesulfonic acid, and p-toluenesulfonic acid. Common inorganic alkalies include (but are not limited to) sodium hydroxide, potassium hydroxide, calcium hydroxide, and barium hydroxide. Common organic alkalies include (but are not limited to) diethylamine, triethylamine, and ethambutol.

The term “stereoisomer” (or “optical isomer”) refers to a stable isomer that has at least one chiral factor (including chiral center, chiral axis, chiral plane) resulting in a perpendicular asymmetric plane and can rotate plane polarized light. Since the compounds of the present disclosure may contain asymmetric centers and other chemical structures that may lead to stereoisomerism, the present disclosure also includes these stereoisomers and mixtures thereof. Since the compounds of the present disclosure and their salts include an asymmetric carbon atom, they can exist in the form of single stereoisomers, racemates, enantiomers, and mixtures of diastereomers. Typically, these compounds can be prepared as racemic mixtures. However, if desired, such compounds can be prepared or isolated as pure stereoisomers, i.e., single enantiomers or diastereomers, or mixtures enriched in a single stereoisomer (with a purity ≥98%, ≥95%, ≥93%, ≥90%, ≥88%, ≥85%, or ≥80%). A single stereoisomer of a compound is prepared synthetically from an optically active starting material containing the desired chiral center, or by preparing a mixture of enantiomeric products followed by separation or resolution. For example, conversion to a mixture of diastereoisomers followed by separation or recrystallization, chromatography, use of a chiral resolving agent, or direct separation of the enantiomers on a chiral chromatographic column. Starting compounds of particular stercochemistry are either commercially available or prepared as described below and resolved by methods well known in the art.

The term “tautomer” (or “tautomeric form”) refers to structural isomers of different energies which are interconvertible via a low energy barrier. If tautomerism is possible (such as in solution), a chemical equilibrium of the tautomers may be achieved. For example, proton tautomers include, but are not limited to, interconversions via migration of a proton, such as keto-enol isomerizations, imine-enamine isomerizations, and amide-iminol isomerizations. Unless otherwise stated, all tautomeric forms of the compounds of the present disclosure are within the scope of the present disclosure.

The term “solvate” refers to a substance formed by the combination of a compound of the present disclosure or a pharmaceutically acceptable salt thereof and at least one solvent molecule through non-covalent intermolecular forces. Common solvates include, but are not limited to, hydrates, ethanolates, and acetoneates.

The term “chelate” refers to a complex having a ring structure, which is obtained by chelation of two or more ligands with the same metal ion to form a chelate ring.

The term “non-covalent complex” is formed by the interaction of a compound with another molecule, where no covalent bond is formed between the compound and the molecule. For example, complexing can occur through van der Waals interaction, hydrogen bonding, and electrostatic interactions (also known as ionic bonding).

The term “independently” means that at least two groups (or ring systems) with the same or similar value ranges in the structure may have the same or different meanings in specific circumstances. For example, the substituent R₁ and the substituent R₂ are independently dodecane-1-yl, octadecane-1-yl, and tetracosane-1-yl. For example, when the substituent R₁ is dodecane-1-yl, the substituent R₂ can be dodecane-1-yl, octadecane-1-yl, and tetracosane-1-yl; similarly, when the substituent R₂ is dodecane-1-yl, the substituent R₁ can also be dodecane-1-yl, octadecane-1-yl, and tetracosane-1-yl.

The term “optional” or “optionally” means that the subsequently described event or circumstance may or may not occur, and that the description includes both the occurrence of said event or circumstance and the non-occurrence of said event or circumstance.

The terms “including” and “comprising” are used in their open, non-limiting sense.

The term “alkyl” refers to a monovalent straight or branched chain alkane radical consisting solely of carbon and hydrogen atoms, containing no unsaturation, and ligated to other moieties by a single bond, including but not limited to methyl, ethyl, propyl, isopropyl, butyl, sec-butyl, isobutyl, and tert-butyl. For example, “C₁₋₂₄ alkyl” refers to a saturated monovalent straight or branched hydrocarbon group containing 1 to 24 carbon atoms.

To provide clearer understanding of the technical solutions of the present disclosure for those skilled in the art, the technical solutions in the present disclosure will be described in detail below in conjunction with specific examples.

The experimental methods described in the following examples are conventional methods unless otherwise specified; and the reagents and equipment can be obtained from commercial sources unless otherwise specified.

In the present disclosure, the “equivalent (eq)” ratio refers to the molar ratio of the solvent or the drug.

In the present disclosure, “appropriate amount” means that the amount of the added solvent or the amount of the drug can be adjusted in a wide range and has little effect on the synthesis result, and no specific limitation is made.

In the following examples, all solvents and drugs used are analytically pure or chemically pure; the solvents are redistilled before use; the anhydrous solvents are treated according to standard methods or literature methods.

### Example 1: Synthesis of Compound 1

Lipoic acid (1.0 eq) was dissolved in an appropriate amount of chloroform and stirred to dissolve. Carbonyl diimidazole (1.1 eq) was added and stirred at room temperature in the dark for 30 min. 1,2-heptanoylphosphatidylethanolamine (0.8 eq) was added and reacted at 30° C. in the dark for 24 h. After the reaction was completed, the chloroform was removed by rotary evaporation, and the compound 1 was obtained by column chromatography:

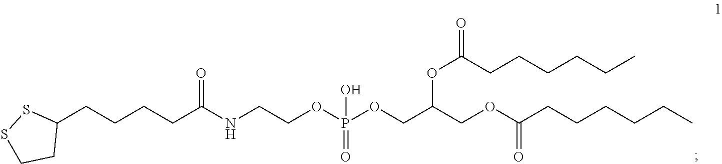 [SMILES: CCCCCCC(=O)OCC(COP(=O)(O)OCCNC(=O)CCCCC1CCSS1)OC(=O)CCCCCC]

¹H NMR (400 MHZ, CDCl3): δ0.86-0.90 (t, 6H), 1.27-1.30 (d, 14H), 1.51-1.66 (m, 8H), 1.73 (s, 1H), 1.97 (s, 1H), 2.01-2.36 (m, 8H), 2.59 (s, 1H), 3.46 (t, 2H), 4.17-4.38 (m, 6H), 5.27 (m, 1H), 6.26 (s, 1H).

### Example 2: Synthesis of Compound 2

Lipoic acid (1.0 eq) was dissolved in an appropriate amount of chloroform and stirred to dissolve. Carbonyl diimidazole (1.1 eq) was added and stirred at room temperature in the dark for 30 min. 1,2-octanoylphosphatidylethanolamine (0.8 eq) was added and reacted at 30° C. in the dark for 24 h. After the reaction was completed, the chloroform was removed by rotary evaporation, and the compound 2 was obtained by column chromatography:

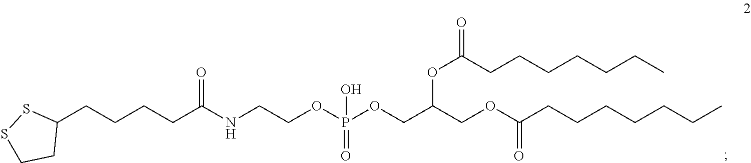 [SMILES: CCCCCCCC(=O)OCC(COP(=O)(O)OCCNC(=O)CCCCC1CCSS1)OC(=O)CCCCCCC]

¹H NMR (400 MHz, CDCl₃): δ0.83-0.92 (t, 6H), 1.25-1.31 (d, 18H), 1.46-1.62 (m, 8H), 1.70 (s, 1H), 1.93 (s, 1H), 2.05-2.32 (m, 8H), 2.66 (s, 1H), 3.36 (t, 2H), 4.15-4.31 (m, 6H), 5.37 (m, 1H), 6.86 (s, 1H).

### Example 3: Synthesis of Compound 3

Lipoic acid (1.0 eq) was dissolved in an appropriate amount of chloroform and stirred to dissolve. Carbonyl diimidazole (1.1 eq) was added and stirred at room temperature in the dark for 30 min. 1,2-nonanoylphosphatidylethanolamine (0.8 eq) was added and reacted at 30° C. in the dark for 24 h. After the reaction was completed, the chloroform was removed by rotary evaporation, and the compound 3 was obtained by column chromatography:

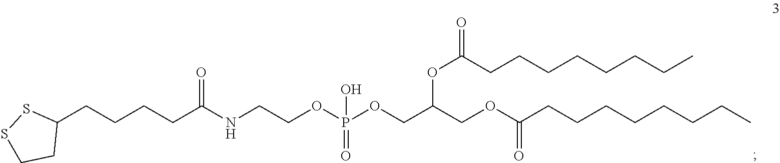 [SMILES: CCCCCCCCC(=O)OCC(COP(=O)(O)OCCNC(=O)CCCCC1CCSS1)OC(=O)CCCCCCCC]

¹H NMR (400 MHZ, CDCl3): δ0.82-0.96 (t, 6H), 1.22-1.36 (d, 22H), 1.45-1.58 (m, 8H), 1.67 (s, 1H), 1.83 (s, 1H), 2.12-2.37 (m, 8H), 2.62 (s, 1H), 3.41 (t, 2H), 4.21-4.37 (m, 6H), 5.27 (m, 1H), 5.86 (s, 1H).

### Example 4: Synthesis of Compound 4

Lipoic acid (1.0 eq) was dissolved in an appropriate amount of chloroform and stirred to dissolve. Carbonyl diimidazole (1.1 eq) was added and stirred at room temperature in the dark for 30 min. 1,2-dodecylphosphatidylethanolamine (0.8 eq) was added and reacted at 30° C. in the dark for 24 h. After the reaction was completed, the chloroform was removed by rotary evaporation, and the compound 4 was obtained by column chromatography:

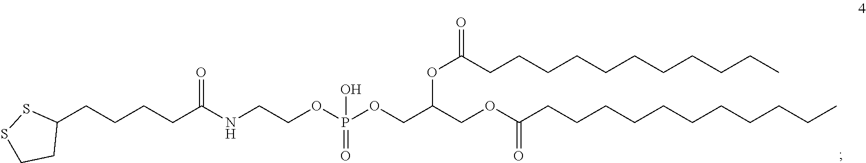 [SMILES: CCCCCCCCCCCC(=O)OCC(COP(=O)(O)OCCNC(=O)CCCCC1CCSS1)OC(=O)CCCCCCCCCCC]

¹H NMR (400 MHZ, CDCl3): δ0.85-0.96 (t, 6H), 1.22-1.36 (d, 34H), 1.45-1.58 (m, 8H), 1.67 (s, 1H), 1.83 (s, 1H), 2.12-2.37 (m, 8H), 2.62 (s, 1H), 3.41 (t, 2H), 4.21-4.37 (m, 6H), 5.27 (m, 1H), 5.86 (s, 1H).

### Example 5: Synthesis of Compound 5

Lipoic acid (1.0 eq) was dissolved in an appropriate amount of chloroform and stirred to dissolve. Carbonyl diimidazole (1.1 eq) was added and stirred at room temperature in the dark for 30 min. 1,2-tetradecanoylphosphatidylethanolamine (0.8 eq) was added and reacted at 30° C. in the dark for 24 h. After the reaction was completed, the chloroform was removed by rotary evaporation, and the compound 5 was obtained by column chromatography:

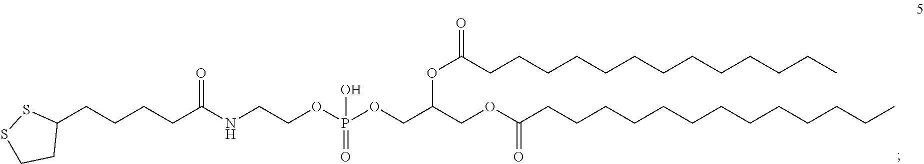 [SMILES: CCCCCCCCCCCCCC(=O)OCC(COP(=O)(O)OCCNC(=O)CCCCC1CCSS1)OC(=O)CCCCCCCCCCCCC]

1H NMR (400 MHz, CDCl3): δ0.87-0.93 (t, 6H), 1.24-1.32 (d, 42H), 1.40-1.52 (m, 8H), 1.63 (s, 1H), 1.73 (s, 1H), 2.16-2.31 (m, 8H), 2.52 (s, 1H), 3.46 (t, 2H), 4.26-4.32 (m, 6H), 5.24 (m, 1H), 5.66 (s, 1H).

### Example 6: Synthesis of Compound 6

Lipoic acid (1.0 eq) was dissolved in an appropriate amount of chloroform and stirred to dissolve. Carbonyl diimidazole (1.1 eq) was added and stirred at room temperature in the dark for 30 min. Dipalmitoylphosphatidylethanolamine (0.8 eq) was added and reacted at 30° C. in the dark for 24 h. After the reaction was completed, the chloroform was removed by rotary evaporation, and the compound 6 was obtained by column chromatography:

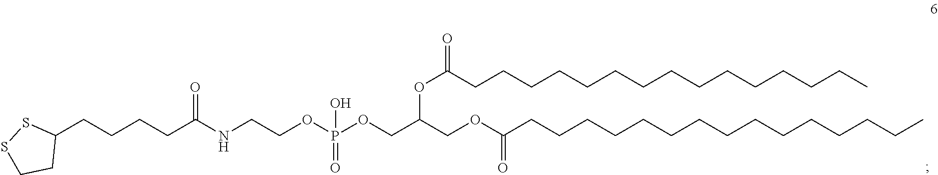 [SMILES: CCCCCCCCCCCCCCCC(=O)OCC(COP(=O)(O)OCCNC(=O)CCCCC1CCSS1)OC(=O)CCCCCCCCCCCCCCC]

¹H NMR (400 MHZ, CDCl3): δ0.82-0.87 (t, 6H), 1.27-1.36 (d, 50H), 1.46-1.57 (m, 8H), 1.66 (s, 1H), 1.75 (s, 1H), 2.23-2.37 (m, 8H), 2.58 (s, 1H), 3.44 (t, 2H), 4.23-4.37 (m, 6H), 5.26 (m, 1H), 5.54 (s, 1H).

### Example 7: Synthesis of Compound 7

Lipoic acid (1.0 eq) was dissolved in an appropriate amount of chloroform and stirred to dissolve. Carbonyl diimidazole (1.1 eq) was added and stirred at room temperature in the dark for 30 min. 1-palmitoyl-2-oleoylethanolamine (0.8 eq) was added and reacted at 30° C. in the dark for 24 h. After the reaction was completed, the chloroform was removed by rotary evaporation, and the compound 7 was obtained by column chromatography:

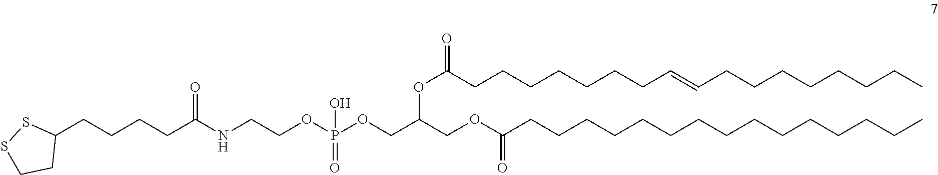 [SMILES: CCCCCCCC/C=C/CCCCCCCC(=O)OC(COC(=O)CCCCCCCCCCCCCCC)COP(=O)(O)OCCNC(=O)CCCCC1CCSS1]

¹H NMR (400 MHZ, CDCl3): δ0.83-0.87 (t, 6H), 1.21-1.36 (d, 48H), 1.43-1.58 (m, 8H), 1.66 (s, 1H), 1.78 (s, 1H), 2.19-2.40 (m, 8H), 2.56 (s, 1H), 3.49 (t, 2H), 4.23-4.30 (m, 6H), 5.24 (m, 1H), 5.45 (m, 2H), 5.67 (s, 1H).

### Example 8: Synthesis of Compound 8

Lipoic acid (1.0 eq) was dissolved in an appropriate amount of chloroform and stirred to dissolve. Carbonyl diimidazole (1.1 eq) was added and stirred at room temperature in the dark for 30 min. 1,2-dioleoyl-SN-glycero-3-phosphoethanolamine (0.8 eq) was added and reacted at 30° C. in the dark for 24 h. After the reaction was completed, the chloroform was removed by rotary evaporation, and the compound 8 was obtained by column chromatography:

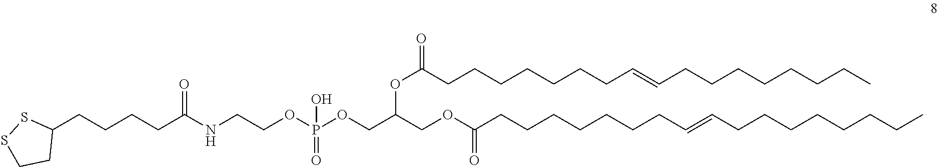 [SMILES: CCCCCCCC/C=C/CCCCCCCC(=O)OCC(COP(=O)(O)OCCNC(=O)CCCCC1CCSS1)OC(=O)CCCCCCC/C=C/CCCCCCCC]

¹H NMR (400 MHZ, CDCl3): δ0.86-0.89 (t, 6H), 1.22-1.36 (d, 48H), 1.40-1.52 (m, 8H), 1.63 (s, 1H), 1.73 (s, 1H), 2.16-2.31 (m, 8H), 2.52 (s, 1H), 3.46 (t, 2H), 4.26-4.32 (m, 6H), 5.29 (m, 1H), 5.65 (m, 4H), 5.86 (s, 1H).

### Example 9: Synthesis of Compound 9

Lipoic acid (1.0 eq) was dissolved in an appropriate amount of chloroform and stirred to dissolve. Carbonyl diimidazole (1.1 eq) was added and stirred at room temperature in the dark for 30 min. 1-stearoyl-2-linoleic acid-sn-glycero-3-phosphoethanolamine (0.8 eq) was added and reacted at 30° C. in the dark for 24 h. After the reaction was completed, the chloroform was removed by rotary evaporation, and the compound 9 was obtained by column chromatography:

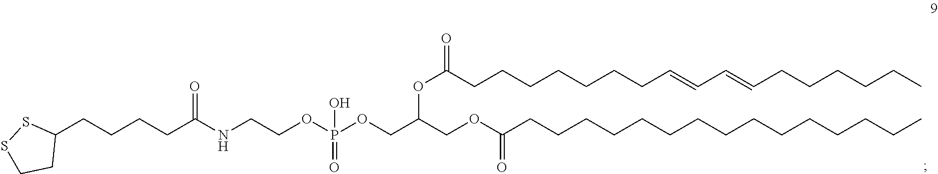 [SMILES: CCCCCC/C=C/C=C/CCCCCCCC(=O)OC(COC(=O)CCCCCCCCCCCCCCC)COP(=O)(O)OCCNC(=O)CCCCC1CCSS1]

¹H NMR (400 MHz, CDCl₃): δ0.83-0.87 (t, 6H), 1.24-1.32 (d, 50H), 1.44-1.56 (m, 8H), 1.67 (s, 1H), 1.72 (s, 1H), 2.14-2.36 (m, 8H), 2.53 (s, 1H), 3.42 (t, 2H), 4.24-4.36 (m, 6H), 5.16 (m, 1H), 5.62 (m, 2H), 5.72 (s, 1H).

### Example 10: Synthesis of Compound 10

2-(1,2-disulfan-3-yl)acetic acid (1.0 eq) was dissolved in an appropriate amount of chloroform and stirred to dissolve. Carbonyl diimidazole (1.1 eq) was added and stirred at room temperature in the dark for 30 min. 1,2-heptanoylphosphatidylethanolamine (0.8 eq) was added and reacted at 30° C. in the dark for 24 h. After the reaction was completed, the chloroform was removed by rotary evaporation, and the compound 10 was obtained by column chromatography:

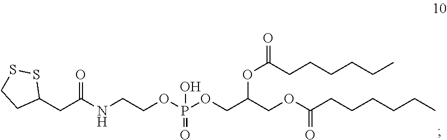 [SMILES: CCCCCCC(=O)OCC(COP(=O)(O)OCCNC(=O)CC1CCSS1)OC(=O)CCCCCC]

¹H NMR (400 MHz, CDCl3): δ0.82-0.88 (t, 6H), 1.31-1.33 (d, 24H), 1.54-1.66 (m, 4H), 1.74 (s, 1H), 1.96 (s, 1H), 2.33-2.54 (m, 5H), 3.46-3.48 (s, 2H), 4.12-4.36 (m, 6H), 5.26-5.28 (m, 1H), 5.62 (m, 2H).

### Example 11: Synthesis of Compound 11

2-(1,2-disulfan-3-yl)propanoic acid (1.0 eq) was dissolved in an appropriate amount of chloroform and stirred to dissolve. Carbonyl diimidazole (1.1 eq) was added and stirred at room temperature in the dark for 30 min. 1-stearoyl-2-linoleic acid-sn-glycero-3-phosphoethanolamine (0.8 eq) was added and reacted at 30° C. in the dark for 24 h. After the reaction was completed, the chloroform was removed by rotary evaporation, and the compound 11 was obtained by column chromatography:

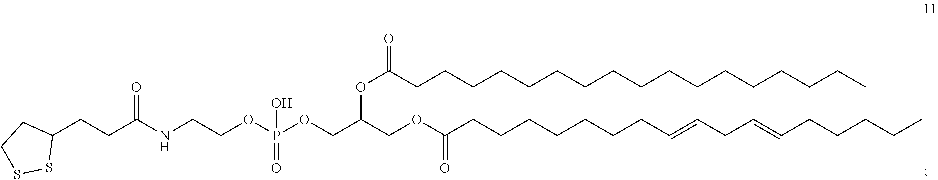 [SMILES: CCCCC/C=C/C/C=C/CCCCCCCC(=O)OCC(COP(=O)(O)OCCNC(=O)CCC1CCSS1)OC(=O)CCCCCCCCCCCCCCCCC]

¹H NMR (400 MHZ, CDCl3): δ0.82-0.85 (t, 6H), 1.22-1.30 (d, 46H), 1.41-1.52 (m, 8H), 1.66 (s, 1H), 1.70 (s, 1H), 2.11-2.32 (m, 8H), 2.51 (s, 1H), 3.46 (t, 2H), 4.27-4.34 (m, 6H), 5.18 (m, 1H), 5.62 (m, 2H), 5.81 (s, 1H).

### Example 12: Synthesis of Compound 12

2-(1,2-disulfan-3-yl)pentanoic acid (1.0 eq) was dissolved in an appropriate amount of chloroform and stirred to dissolve. Carbonyl diimidazole (1.1 eq) was added and stirred at room temperature in the dark for 30 min. Dioleoylphosphatidyl-glycero-3-phosphoethanolamine (0.8 eq) was added and reacted at 30° C. in the dark for 24 h. After the reaction was completed, the chloroform was removed by rotary evaporation, and the compound 12 was obtained by column chromatography:

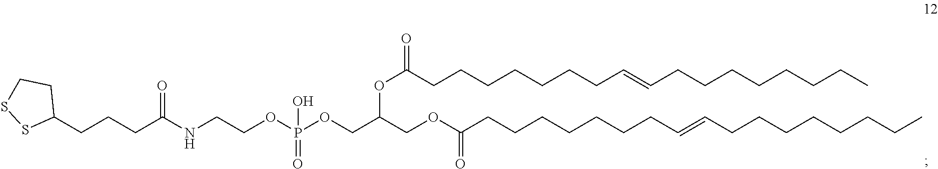 [SMILES: CCCCCCCC/C=C/CCCCCCCC(=O)OCC(COP(=O)(O)OCCNC(=O)CCCC1CCSS1)OC(=O)CCCCCCC/C=C/CCCCCCCC]

¹H NMR (400 MHz, CDCl3): δ0.85-0.88 (t, 6H), 1.21-1.37 (d, 46H), 1.38-1.53 (m, 8H), 1.61 (s, 1H), 1.70 (s, 1H), 2.15-2.33 (m, 8H), 2.50 (s, 1H), 3.41 (t, 2H), 4.20-4.27 (m, 6H), 5.25 (m, 1H), 5.65 (m, 4H), 5.76 (s, 1H).

### Example 13: Synthesis of LNPs

Compounds 7, 8, 9, 10, cholesterol, ALC-0315 (((4-hydroxybutyl)azadiyl)bis(hexane-6,1-diyl)bis(2-hexyldecanoate)), and PEG-DSPE (polyethylene glycol-distearoylphosphoethanolamine) were dissolved in ethanol at a molar ratio of 10:38:49:3 (based on the total weight of lipids, to obtain a lipid solution with a concentration of 20 mg/mL), PBS was used as an aqueous phase, and a volume ratio of the two solutions was 1:3 (where the equivalent of the ethanol solution was 1 and the equivalent of the aqueous solution was 3). The two phases were quickly mixed using microfluidic technology and dialyzed to remove the ethanol to obtain LNPs.

### Example 14: Preparation of the Nucleic Acid-LNP Composition

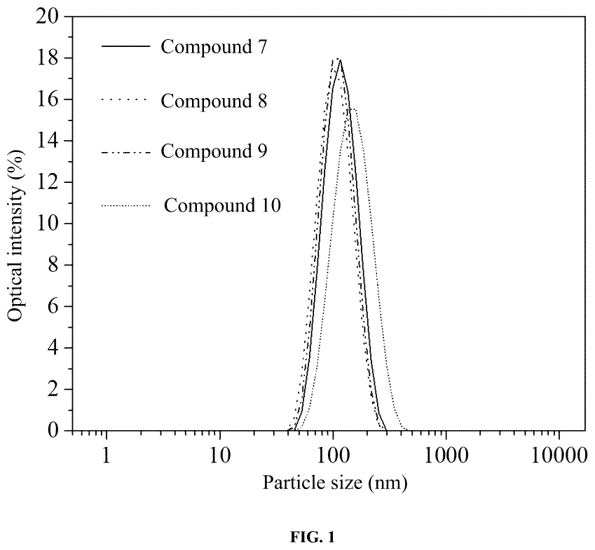

Compounds 7, 8, 9, 10, cholesterol, ALC-0315 (((4-hydroxybutyl)azadiyl)bis(hexane-6,1-diyl)bis(2-hexyldecanoate)), and PEG-DSPE (polyethylene glycol-distearoylphosphoethanolamine) were dissolved in ethanol at a molar ratio of 10:38:49:3 (based on the total weight of lipids, to obtain a lipid solution with a concentration of 30 mg/mL), siPLK1 small interfering RNA was dissolved in 10 mM citrate buffered saline solution at pH=4.0, PBS was used as an aqueous phase, and a volume ratio of the two solutions was 1:3 (where the equivalent of the ethanol solution was 1 and the equivalent of the aqueous solution was 3). The two phases were quickly mixed using microfluidic technology and dialyzed to replace the buffer environment with PBS at pH=7.4, so as to remove ethanol to obtain the nucleic acid-LNP composition. As shown in FIG. 1, the nucleic acid-LNP compositions prepared based on compounds 7, 8, 9, and 10 had similar particle size distributions.

### Example 15: Preparation of Nucleic Acid-LNP Pharmaceutical Preparation

Compounds 7, 8, 9, 10, cholesterol, ALC-0315 (((4-hydroxybutyl)azadiyl)bis(hexane-6,1-diyl)bis(2-hexyldecanoate)), and PEG-DSPE (polyethylene glycol-distearoylphosphoethanolamine) were dissolved in ethanol at a molar ratio of 10:38:49:3 (based on the total weight of lipids, to obtain a lipid solution with a concentration of 20 mg/mL), cGFP plasmid was dissolved in 10 mM citrate buffered saline solution at pH=4.0, PBS was used as an aqueous phase, and a volume ratio of the two solutions was 1:3 (where the equivalent of the ethanol solution was 1 and the equivalent of the aqueous solution was 3). The two phases were quickly mixed using impact mixer and dialyzed to replace the buffer environment with PBS at pH=7.4, sodium chloride was added to adjust the osmotic pressure, and ethyl cellulose was added as a suspending agent to obtain the nucleic acid-LNP pharmaceutical preparation.

### Example 16: Preparation of Nucleic Acid-LNP Pharmaceutical Preparation

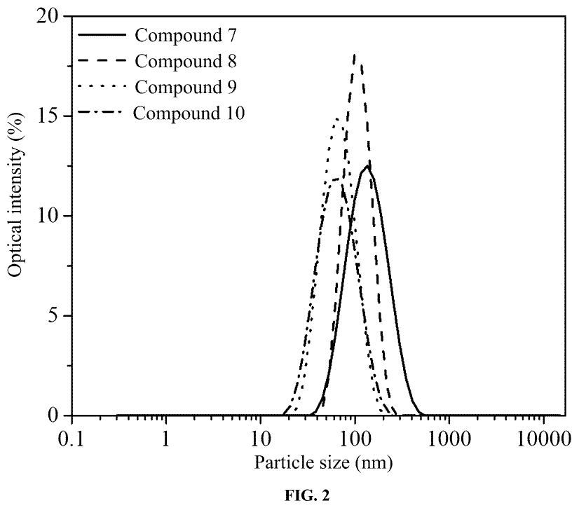

Compounds 7, 8, 9, 10, cholesterol, ALC-0315 (((4-hydroxybutyl)azadiyl)bis(hexane-6,1-diyl)bis(2-hexyldecanoate)), and PEG-DSPE (polyethylene glycol-distearoylphosphoethanolamine) were dissolved in ethanol at a molar ratio of 10:38:49:3 (based on the total weight of lipids, to obtain a lipid solution with a concentration of 20 mg/mL), siPLK1 small interfering RNA was dissolved in 10 mM citrate buffered saline solution at pH=4.0, PBS was used as an aqueous phase, and a volume ratio of the two solutions was 1:3 (where the equivalent of the ethanol solution was 1 and the equivalent of the aqueous solution was 3). The two phases were quickly mixed using microfluidic technology and dialyzed to replace the buffer environment with PBS at pH=7.4, glycerol was added to adjust the osmotic pressure and hydroxypropyl methylcellulose was added as a suspending agent to obtain a nucleic acid-LNP pharmaceutical preparation, and the particle size distribution was shown in FIG. 2.

### Example 17: Preparation of Nucleic Acid-LNP Pharmaceutical Preparation

Compounds 7, 8, 9, 10, cholesterol, ALC-0315 (((4-hydroxybutyl)azadiyl)bis(hexane-6,1-diyl)bis(2-hexyldecanoate)), and PEG-DSPE (polyethylene glycol-distearoylphosphoethanolamine) were dissolved in ethanol at a molar ratio of 10:38:49:3 (based on the total weight of lipids, to obtain a lipid solution with a concentration of 20 mg/mL), cGFP plasmid was dissolved in 10 mM citrate buffered saline solution at pH=4.0, PBS was used as an aqueous phase, and a volume ratio of the two solutions was 1:3 (where the equivalent of the ethanol solution was 1 and the equivalent of the aqueous solution was 3). The two phases were quickly mixed using microjet and dialyzed to replace the buffer environment with HEPES buffer at pH=7.4, glucose was added to adjust the osmotic pressure, and polyvinyl alcohol was added as a suspending agent to obtain the nucleic acid-LNP pharmaceutical preparation.

### Example 18: Laser Confocal Microscopy of LNPs

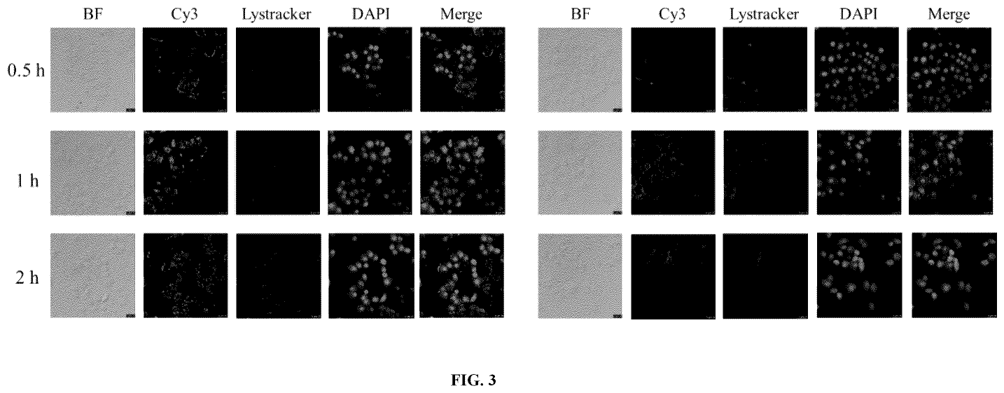

Compound 7, cholesterol, ALC-0315 (((4-hydroxybutyl)azadiyl)bis(hexane-6,1-diyl)bis(2-hexyldecanoate)), and Cy3-PEG-DSPE (polyethylene glycol-distearoylphosphoethanolamine) were dissolved in ethanol at a molar ratio of 10:38:49:3 (based on the total weight of lipids, to obtain a lipid solution with a concentration of 20 mg/mL), PBS was used as an aqueous phase, and a volume ratio of the two solutions was 1:3 (where the equivalent of the ethanol solution was 1 and the equivalent of the aqueous solution was 3). The two phases were quickly mixed using microfluidic technology and dialyzed to remove the ethanol to obtain LNPs 1. The compound 7 in the above experiment was replaced with DSPC, and LNPs 2 were prepared using the same formulation and process. After the two LNPs were incubated with A549 cells for 0.5, 1, and 2 h, the samples were washed with PBS, and lysosomes were stained with Lystracker-green for 1 h. The cells were then fixated with 4% paraformaldehyde, and finally the cell nuclei were stained with DAPI. Laser confocal microscopy was conducted to observe the cell bright field, nucleus, lysosomes, and samples. It was observed by confocal microscopy that the lipid containing dithiolane structure designed by the present disclosure could promote the endocytosis of LNPs by A549 cells and had a lysosomal escape effect. The laser confocal image was shown in FIG. 3. The LNPs were labeled with Cy3 and their distribution in A549 cells was observed. Meanwhile, cells were stained with DAPI and lysosomes labeled with Lysotracker green were used as reference. The left figure showed LNPs 1, and the right figure showed LNPs 2. As shown in FIG. 3, black was the cell nucleus stained with DAPI, dark gray was the LNP labeled with Cy3, and light gray was the lysosome labeled with Lysotracker green. LNP with dithiolane could significantly improve the ability of LNP to enter cells. From the confocal results, LNP was distributed around the A549 cell nucleus, indicating that A549 cells could effectively internalize LNP and had a certain lysosomal escape ability.

### Example 19: Transfection Experiment of cGFP LNPs

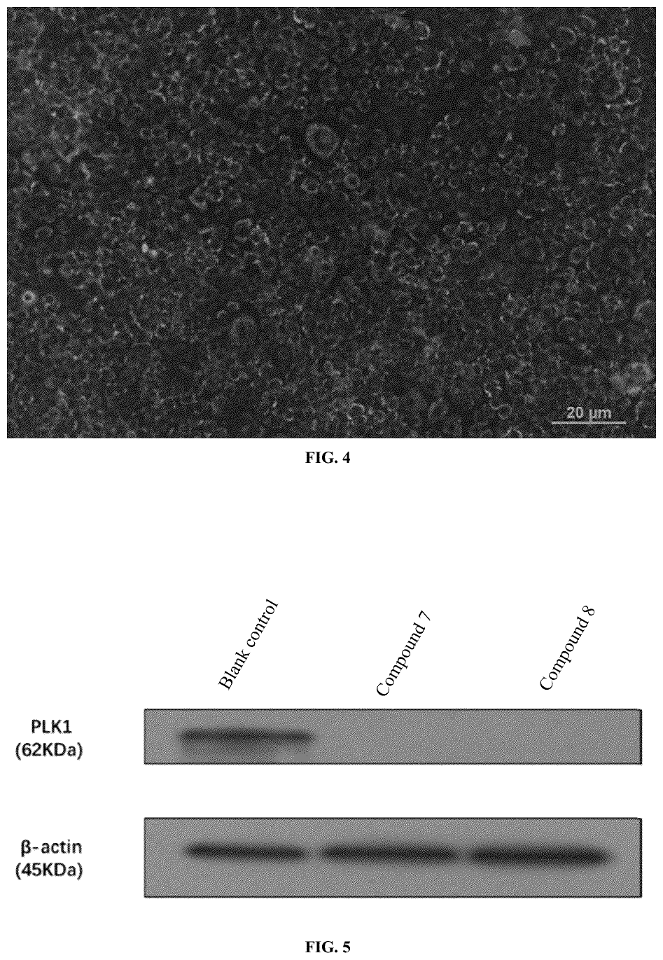

Compound 7, cholesterol, ALC-0315 (((4-hydroxybutyl)azadiyl)bis(hexane-6,1-diyl)bis(2-hexyldecanoate)), and Cy3-PEG-DSPE (polyethylene glycol-distearoylphosphoethanolamine) were dissolved in ethanol at a molar ratio of 10:38:49:3 (based on the total weight of lipids, to obtain a lipid solution with a concentration of 20 mg/mL), aqueous phase was 100 mM citric acid at pH=4.0, and the concentration of cGFP was 0.2 mg/mL, and a volume ratio of the two solutions was 1:3 (where the equivalent of the ethanol solution was 1 and the equivalent of the aqueous solution was 3). The two phases were quickly mixed using microfluidic technology and dialyzed to remove the ethanol to obtain eGFP-encapsulated LNPs. The eGFP-encapsulated LNPs were incubated with A549 cells for 1 h, then washed with PBS to remove the sample, and the fluorescence in the cells was observed under a fluorescence microscope. It was seen through the fluorescence microscope that almost all A549 cells were successfully transferred with the cGFP plasmid, and the fluorescence image was shown in FIG. 4.

### Example 20: Protein Inhibition Experiment of siPLK1 LNPs

Compound 7, cholesterol, ALC-0315 (((4-hydroxybutyl)azadiyl)bis(hexane-6,1-diyl)bis(2-hexyldecanoate)), and Cy3-PEG-DSPE (polyethylene glycol-distearoylphosphoethanolamine) were dissolved in ethanol at a molar ratio of 10:38:49:3 (based on the total weight of lipids, to obtain a lipid solution with a concentration of 20 mg/mL), aqueous phase was 100 mM citric acid at pH=4.0, and the concentration of siPLK1 was 0.2 mg/mL, and a volume ratio of the two solutions was 1:3 (where the equivalent of the ethanol solution was 1 and the equivalent of the aqueous solution was 3). The two phases were quickly mixed using microfluidic technology and dialyzed to remove the ethanol to obtain siPLK1-encapsulated LNPs. The compound 7 in the above experiment was replaced with DSPC, and LNPs 2 were prepared using the same formulation and process. The two types of siPLK1-encapsulated LNPs were incubated with A549 cells for 1 h and then washed with PBS. The PLK1 protein in the cells was detected by protein electrophoresis using β-actin protein as a reference. From the protein electrophoresis results, it was seen that the siPLK1-encapsulated LNPs could inhibit the expression of PLK1 protein in A549 cells, and the protein electrophoresis image was shown in FIG. 5.

### Example 21: Preparation of TEM Samples of siPLK1 LNPs

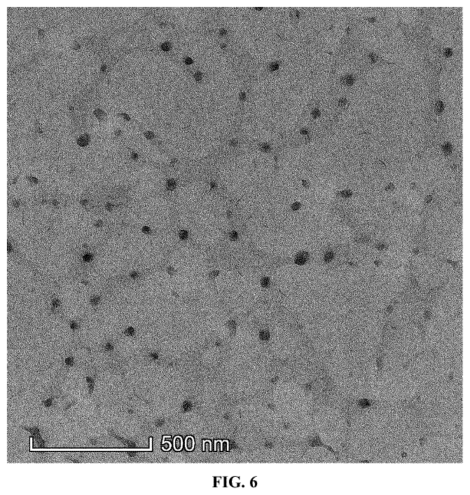

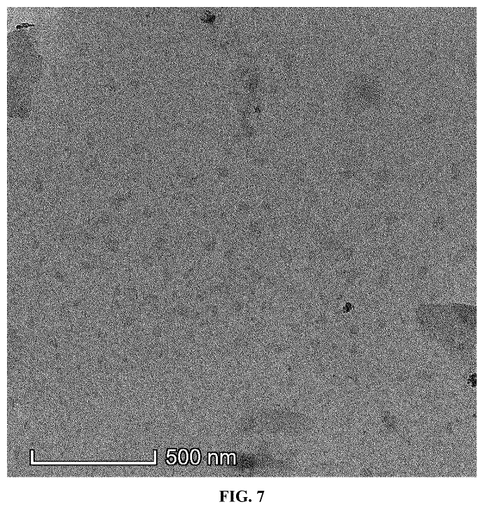

Compound 7, cholesterol, ALC-0315 (((4-hydroxybutyl)azadiyl)bis(hexane-6,1-diyl)bis(2-hexyldecanoatc)), and Cy3-PEG-DSPE (polyethylenc glycol-distearoylphosphoethanolamine) were dissolved in ethanol at a molar ratio of 10:38:49:3 (based on the total weight of lipids, to obtain a lipid solution with a concentration of 20 mg/mL), aqueous phase was 100 mM citric acid at pH=4.0, and the concentration of siPLK1 was 0.2 mg/mL, and a volume ratio of the two solutions was 1:3 (where the equivalent of the ethanol solution was 1 and the equivalent of the aqueous solution was 3). The two phases were quickly mixed using microfluidic technology and dialyzed to remove the ethanol to obtain siPLK1-encapsulated LNPs. The compound 7 in the above experiment was replaced with DSPC, and LNPs 2 were prepared using the same formulation and process. The two prepared LNPs were dropped on 2 electron microscope copper grids, allowed to stand for 3 min, and the liquid was absorbed from the back of the copper grid using filter paper. Then a drop of phosphotungstic acid dye solution was dropped on the copper grid, allowed to stand for 3 min, and the liquid was absorbed from the back of the copper grid using filter paper. Finally, a drop of purified water was dropped on the copper grid, and the liquid was absorbed from the back of the copper grid using filter paper to clean the copper grid. The prepared TEM samples were stored away from light and sent for observation, and the electron microscope images were shown in FIG. 4 and FIG. 5. The dithiolane structure could easily complex heavy metal ions. Phosphotungstic acid was used as a stain for TEM to stain the nucleic acid-LNP composition prepared in this example and the nucleic acid-LNP composition prepared using DSPE-PEG-LA material with phosphotungstic acid and then TEM photos were taken. As shown in FIG. 6, the LNP of this example was clearly stained in the electron microscope photograph due to the complexation of phosphotungstic acid. As shown in FIG. 7, the electron microscope photograph of the LNP prepared using the DSPE-PEG-LA material was darker because less dithiolane was exposed on the LNP surface.

The above description is merely preferred examples of the present disclosure and is not intended to limit the present disclosure, and various changes and modifications of the present disclosure may be made by those skilled in the art. Any modifications, equivalent substitutions, improvements, and the like made within the spirit and principle of the present disclosure should be included within the protection scope of the present disclosure.

What is claimed is:

1. A glycerylphosphorylethanolamine (GPE) skeleton compound having a structure shown in formula (I): 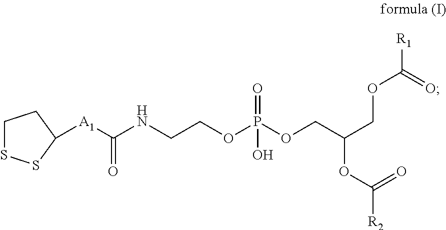
wherein
R₁ and R₂ are independently selected from the group consisting of C₁₋₂₄ alkyl and C₁₋₂₄ alkenyl, and A₁ is selected from the group consisting of methylene, 1,2-ethanediyl, 1,3-propylenediyl, 1-methyl-1,2-ethanediyl, 1,2-isopropyl, 1-methyl-1,3-propylenediyl, 2-methyl-1,3-propylenediyl, 1,4-butanediyl, 1,5-pentanediyl, 1-methyl-1,4-butanediyl, 2-methyl-1,4-butanediyl, 3-methyl-1,4-butanediyl, 2,2-dimethyl-1,3-butanediyl, 1,6-hexanediyl, 1-methyl-1,5-pentanediyl, 2-methyl-1,5-pentanediyl, 3-methyl-1,5-pentanediyl, 1-ethyl-1,4-butanediyl, and 2-ethyl-1,4-butanediyl.

2. A preparation method of the GPE skeleton compound according to claim 1, comprising the following steps:
dissolving a compound shown in formula (II) in chloroform with stirring, adding carbonyl diimidazole, stirring an obtained first mixture in the dark, adding a compound shown in formula (III), subjecting an obtained second mixture to a reaction in the dark, subjecting an obtained product to rotary evaporation to remove the chloroform, and conducting column chromatography to obtain the GPE skeleton compound; wherein

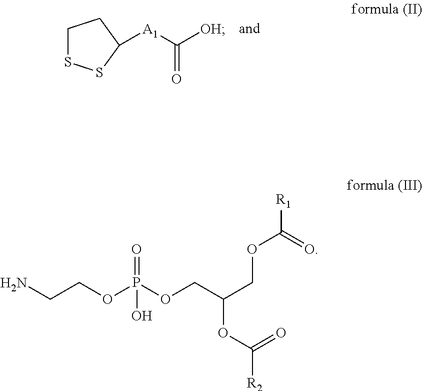

3. The preparation method according to claim 2, wherein the compound shown in formula (II), the carbonyl diimidazole, and the compound shown in formula (III) are at a molar ratio of 1:(0.75-0.85):(1.05-1.15).

4. The preparation method according to claim 2, wherein the reaction is conducted in the dark at 29° C. to 31° C. for 22 h to 26 h.

5. A lipid nanoparticle (LNP)-based drug carrier, comprising a first lipid compound and a second lipid compound, wherein the first lipid compound is one or more selected from the group consisting of the GPE skeleton compound according to claim 1 and a pharmaceutically acceptable salt, a stereoisomer, a tautomer, a solvate, a chelate, and a non-covalent complex thereof, and the second lipid compound is selected from the group consisting of a cationic lipid or an ionizable lipid, a sterol, and an amphiphilic lipid.

6. The LNP-based drug carrier according to claim 5, wherein the cationic lipid or the ionizable lipid is one or more selected from the group consisting of DLinDMA, DODMA, DLin-MC2-MPZ, DLin-KC2-DMA, DOTAP, C12-200, SM-102, and ALC-0315; and
the amphiphilic lipid is one or more selected from the group consisting of PEG-DSPE, PEG-PE, PEG-DMG, PEG-C14, PEG-c-DMA, PEG-modified ceramide, PEG-modified dialkylamine, PEG-modified diacylglycerol, Tween-20, Tween-80, PEG-DPG, PEG-s-DMG, PEG-c-DOMG, and GalNAc-PEG-DSG.

7. The LNP-based drug carrier according to claim 5, wherein the first lipid compound, the cationic lipid or the ionizable lipid, the sterol, and the amphiphilic lipid are at a molar ratio of (2-20):(10-65):(20-55):(0.2-20); and
preferably, the first lipid compound, the cationic lipid or the ionizable lipid, the sterol, and the amphiphilic lipid are at a molar ratio of (5-10):(30-50):(35-55):(0.5-10).

8. A nucleic acid-LNP composition having a particle size of 30 nm to 300 nm and comprising the LNP-based drug carrier according to claim 5 and a nucleic acid drug, wherein the LNP-based drug carrier and the nucleic acid drug are at a mass ratio of 1:(1-20).

9. The nucleic acid-LNP composition according to claim 8, wherein the nucleic acid drug is one or more selected from the group consisting of a DNA, an siRNA, an mRNA, a dsRNA, an antisense nucleic acid, a microRNA, an antisense microRNA, antagomir, a microRNA inhibitor, a microRNA activator, and an immunostimulatory nucleic acid.

10. A pharmaceutical preparation, comprising the nucleic acid-LNP composition according to claim 8, a pharmaceutically acceptable buffer salt, an osmotic pressure regulator, and a suspending agent.

11. The nucleic acid-LNP composition according to claim 8, wherein the cationic lipid or the ionizable lipid is one or more selected from the group consisting of DLinDMA, DODMA, DLin-MC2-MPZ, DLin-KC2-DMA, DOTAP, C12-200, SM-102, and ALC-0315; and
the amphiphilic lipid is one or more selected from the group consisting of PEG-DSPE, PEG-PE, PEG-DMG, PEG-C14, PEG-c-DMA, PEG-modified ceramide, PEG-modified dialkylamine, PEG-modified diacylglycerol, Tween-20, Tween-80, PEG-DPG, PEG-s-DMG, PEG-c-DOMG, and GalNAc-PEG-DSG.

12. The nucleic acid-LNP composition according to claim 8, wherein the first lipid compound, the cationic lipid or the ionizable lipid, the sterol, and the amphiphilic lipid are at a molar ratio of (2-20):(10-65):(20-55):(0.2-20); and
preferably, the first lipid compound, the cationic lipid or the ionizable lipid, the sterol, and the amphiphilic lipid are at a molar ratio of (5-10):(30-50):(35-55):(0.5-10).

13. The nucleic acid-LNP composition according to claim 11, wherein the nucleic acid drug is one or more selected from the group consisting of a DNA, an siRNA, an mRNA, a dsRNA, an antisense nucleic acid, a microRNA, an antisense microRNA, antagomir, a microRNA inhibitor, a microRNA activator, and an immunostimulatory nucleic acid.

14. The nucleic acid-LNP composition according to claim 12, wherein the nucleic acid drug is one or more selected from the group consisting of a DNA, an siRNA, an mRNA, a dsRNA, an antisense nucleic acid, a microRNA, an antisense microRNA, antagomir, a microRNA inhibitor, a microRNA activator, and an immunostimulatory nucleic acid.

15. The pharmaceutical preparation according to claim 10, wherein the cationic lipid or the ionizable lipid is one or more selected from the group consisting of DLinDMA, DODMA, DLin-MC2-MPZ, DLin-KC2-DMA, DOTAP, C12-200, SM-102, and ALC-0315; and
the amphiphilic lipid is one or more selected from the group consisting of PEG-DSPE, PEG-PE, PEG-DMG, PEG-C14, PEG-c-DMA, PEG-modified ceramide, PEG-modified dialkylamine, PEG-modified diacylglycerol, Tween-20, Tween-80, PEG-DPG, PEG-s-DMG, PEG-c-DOMG, and GalNAc-PEG-DSG.

16. The pharmaceutical preparation according to claim 10, wherein the first lipid compound, the cationic lipid or the ionizable lipid, the sterol, and the amphiphilic lipid are at a molar ratio of (2-20):(10-65):(20-55):(0.2-20); and
preferably, the first lipid compound, the cationic lipid or the ionizable lipid, the sterol, and the amphiphilic lipid are at a molar ratio of (5-10):(30-50):(35-55):(0.5-10).

17. A pharmaceutical preparation, comprising the nucleic acid-LNP composition according to claim 9, a pharmaceutically acceptable buffer salt, an osmotic pressure regulator, and a suspending agent.

18. The pharmaceutical preparation according to claim 17, wherein the cationic lipid or the ionizable lipid is one or more selected from the group consisting of DLinDMA, DODMA, DLin-MC2-MPZ, DLin-KC2-DMA, DOTAP, C12-200, SM-102, and ALC-0315; and
the amphiphilic lipid is one or more selected from the group consisting of PEG-DSPE, PEG-PE, PEG-DMG, PEG-C14, PEG-c-DMA, PEG-modified ceramide, PEG-modified dialkylamine, PEG-modified diacylglycerol, Tween-20, Tween-80, PEG-DPG, PEG-s-DMG, PEG-c-DOMG, and GalNAc-PEG-DSG.

19. The pharmaceutical preparation according to claim 17, wherein the first lipid compound, the cationic lipid or the ionizable lipid, the sterol, and the amphiphilic lipid are at a molar ratio of (2-20):(10-65):(20-55):(0.2-20); and
preferably, the first lipid compound, the cationic lipid or the ionizable lipid, the sterol, and the amphiphilic lipid are at a molar ratio of (5-10):(30-50):(35-55):(0.5-10).
